(19) United States
(12) Patent Application Publication
(10) Pub. No.: US 2025/0213164 A1
(43) Pub. Date: Jul. 3, 2025

(54) CATHETER ASSEMBLY TRACKING

(71) Applicant: Boston Scientific Scimed Inc., Maple Grove, MN (US)

(72) Inventors: Sarah Renee Gutbrod, St. Paul, MN (US); Thomas Ferree, Waltham, MA (US); Matthew Ferrante, Cambridge, MA (US); Jackie Leung, Richmond Hill (CA); Prateek Mathur, Toronto (CA)

(21) Appl. No.: 19/000,061

(22) Filed: Dec. 23, 2024

Related U.S. Application Data
(60) Provisional application No. 63/616,270, filed on Dec. 29, 2023.

(51) Int. Cl. A61B 5/287; A61B 5/00; A61B 5/06; A61B 5/367; A61B 34/20

(52) U.S. Cl. CPC A61B 5/287; A61B 5/068; A61B 5/367; A61B 5/6852; A61B 34/20; A61B 2034/2051

(57) ABSTRACT

A system for use with an electrophysiological procedure includes a catheter assembly including a plurality of coaxially disposed catheter elements, the plurality of catheter elements movable with respect to each other along the longitudinal axis. The system further includes a controller configured to generate a first position of the first catheter element based on the first electrical signal and generate a second position of the second catheter element based on the second electrical signal and, in response to a detected longitudinal movement of the second catheter element along the longitudinal axis with respect to the first catheter element, apply a historical correction to the first and second positions based on a previously determined first and second position vector to obtain an initially corrected first and second positions. The control further generates an anatomical map of the electrophysiological procedure with a visualization of the catheter assembly including the longitudinal adjustment.

### CROSS REFERENCE TO RELATED APPLICATIONS

The present applications claims priority to, and the benefit of, U.S. Provisional Patent Application No. 63/616,270, filed Dec. 29, 2023, the entire disclosure of which is incorporated herein by reference.

### TECHNICAL FIELD

The present disclosure relates generally to medical systems and methods for electrophysiological procedures, such as ablating cardiac tissue, in a patient via catheter assemblies. More specifically, the present disclosure relates to medical systems and methods for tracking catheter assemblies within the patient during the electrophysiological procedure.

### BACKGROUND

Electrophysiological procedures involve guiding catheter assemblies into the heart and tracking the location of the catheter assemblies with respect to the heart. Catheter ablation is a minimally invasive electrophysiological procedure to treat a variety of heart conditions such as supraventricular and ventricular arrhythmia. Such procedures can involve the visualization of the heart, heart activity, and the position of the catheter assembly within the heart. A common visualization system involves the use of fluoroscopy, which can expose the patient and clinician to ionizing radiation. Electroanatomical mapping is an alternative visualization technique that does not involve the use of ionizing radiation. Electroanatomical mapping allows a clinician to accurately determine the location of an arrhythmia, define cardiac geometry in three dimensions, delineate areas of anatomic interest, and permit spatial localization of the catheter assembly for positioning and manipulation.

Catheter assemblies include a plurality of catheter elements such as catheters, sheaths, dilators, guidewires, and needles. For instance, a catheter assembly can include a catheter and a sheath. Navigation-enabled catheter assembly elements, such as navigation-enabled catheters, use magnetic fields to track a magnetic sensor within the catheter element with relative accuracy in electroanatomical mapping systems. But not all catheter elements include magnetic sensors. Impedance-based catheter elements, such as catheter sheaths or catheters, use electric fields to track the catheter element with lower cost components such as electrodes but are generally less accurate than navigation-enabled catheter elements in electroanatomical mapping systems. Regardless of whether the catheter element is navigation-enabled or impedance-based, electroanatomical mapping systems use electrical measurements to determine a catheter pose, or the three-dimensional curve of the distal portion of the catheter assembly spanned by electrodes. Because of the inaccuracies in the tracking systems, different catheter elements in a catheter assembly may be rendered in a visualization as displaced or separated from one another even though the catheter assembly incudes one catheter element disposed within another, such as a catheter disposed within a sheath, within the patient.

### SUMMARY

Example 1 is a system for use with an electrophysiological procedure. The system includes a catheter assembly including a plurality of coaxially disposed catheter elements, the plurality of catheter elements including a first catheter element and a second catheter element, the first catheter element forming an elongated lumen defining a longitudinal axis and the second catheter element disposed within the lumen, the first and second catheter elements movable with respect to each other along the longitudinal axis, wherein the first catheter element includes a first tracking sensor configured to generate a first electrical signal and the second catheter element includes a second tracking sensor configured to generate a second electrical signal. A controller is configured to generate a first position of the first catheter element based on the first electrical signal and generate a second position of the second catheter element based on the second electrical signal. In response to a detected longitudinal movement of the second catheter element along the longitudinal axis with respect to the first catheter element: apply a historical correction to the first and second positions based on a previously determined first and second position vector to obtain an initially corrected first and second positions, apply a longitudinal adjustment to the initially corrected first and second positions based on a constraint applied to the detected longitudinal movement, and generate an anatomical map of an organ of the electrophysiological procedure with a visualization of the catheter assembly including the longitudinal adjustment.

Example 2 is the system of example 1, wherein the controller is configured to update the previously determined first and second position vector in response to the detected longitudinal movement of the second catheter element along the longitudinal axis with respect to the first catheter element.

Example 3 is the system of example 2, wherein the updated previously determined first and second position vector is based on a plurality of previously determined first and second position vectors.

Example 4 is the system of any of examples 2 and 3, wherein the update to the previously determined first and second position vector is based on a parameter of the electrophysiological procedure.

Example 5 is the system of any of examples 2-4, wherein the update to the previously determined first and second position vector is based on a time period of the electrophysiological procedure.

Example 6 is the system of any of examples 2-5, wherein the update to the previously determined first and second position vector is based on an anatomical location of the electrophysiological procedure.

Example 7 is the system of example 4, wherein the update to the previously determined first and second position vector is based on a plurality of parameters of the electrophysiological procedure.

Example 8 is the system of any of examples 1-7, wherein the controller is configured to laterally align the first position with the second position based on a lateral displacement and a rotational displacement determined from the first position and the second position.

Example 9 is the system of example 8, wherein the first catheter position includes a first location in space and a first tangent, and the second catheter position includes a second location in space and a second tangent, and wherein the first location and the first tangent are laterally aligned with the second location and the second tangent based on a lateral departure and a rotational deflection determined from the first tangent and the second tangent.

Example 10 is the system of any of examples 1-9, wherein the controller is configured to track the first catheter element via impedance tracking and to track the second catheter element via magnetic tracking.

Example 11 is the system of any of examples 8-10, wherein the controller is configured to laterally align the first position to the second position.

Example 12 is the system of any of examples 1-11, wherein the controller is configured to apply the longitudinal adjustment to the first position with respect to the second position.

Example 13 is the system of any of examples 1-12, wherein the detected longitudinal movement of the second catheter element along the longitudinal axis with respect to the first catheter element is based on an independent parameter that detects the relationship between the sheath and electrodes on the catheter.

Example 14 is the system of claim 13, wherein the independent parameter includes sheath detection mechanism.

Example 15 is the system of any of examples 1-13, wherein the first catheter element includes a catheter sheath.

Example 16 is a system for use with an electrophysiological procedure. The system includes a catheter assembly including a plurality of coaxially disposed catheter elements, the plurality of catheter elements including a first catheter element and a second catheter element, the first catheter element forming an elongated lumen defining a longitudinal axis and the second catheter element disposed within the lumen, the first and second catheter elements movable with respect to each other along the longitudinal axis, wherein the first catheter element includes a first tracking sensor configured to generate a first electrical signal and the second catheter element includes a second tracking sensor configured to generate a second electrical signal. A controller is configured to generate a first position of the first catheter element based on the first electrical signal and generate a second position of the second catheter element based on the second electrical signal. In response to a detected longitudinal movement of the second catheter element along the longitudinal axis with respect to the first catheter element: apply a historical correction to the first and second positions based on a previously determined first and second position vector to obtain an initially corrected first and second positions, apply a longitudinal adjustment to the initially corrected first and second positions based on a constraint applied to the detected longitudinal movement, and generate an anatomical map of an organ of the electrophysiological procedure with a visualization of the catheter assembly including the longitudinal adjustment.

Example 17 is the system of example 16, wherein the controller is configured to update the previously determined first and second position vector in response to the detected longitudinal movement of the second catheter element along the longitudinal axis with respect to the first catheter element.

Example 18 is the system of example 17, wherein the updated previously determined first and second position vector is based on a plurality of previously determined first and second position vectors.

Example 19 is the system of example 17, wherein the update to the previously determined first and second position vector is based on a parameter of the electrophysiological procedure.

Example 20 is the system of example 17, wherein the update to the previously determined first and second position vector is based on a time period of the electrophysiological procedure.

Example 21 is the system of example 17, wherein the update to the previously determined first and second position vector is based on an anatomical location of the electrophysiological procedure.

Example 22 is the system of example 17, wherein the update to the previously determined first and second position vector is based on a plurality of parameters of the electrophysiological procedure.

Example 23 is the system of example 16, wherein the controller is configured to laterally align the first position with the second position based on a lateral displacement and a rotational displacement determined from the first position and the second position.

Example 24 is the system of example 23, wherein the first catheter position includes a first location in space and a first tangent, and the second catheter position includes a second location in space and a second tangent, and wherein the first location and the first tangent are laterally aligned with the second location and the second tangent based on a lateral departure and a rotational deflection determined from the first tangent and the second tangent.

Example 25 is the system of example 16, wherein the controller is configured to track the first catheter element via impedance tracking and to track the second catheter element via magnetic tracking.

Example 26 is the system of example 16, wherein the controller is configured to laterally align the second position to the first position.

Example 27 is the system of example 16, wherein the controller is configured to apply the longitudinal adjustment to the second position with respect to the first position.

Example 28 is the system of example 16, wherein the detected longitudinal movement of the second catheter element along the longitudinal axis with respect to the first catheter element is based on an independent parameter that detects the relationship between the sheath and electrodes on the catheter.

Example 29 is the system of claim 28, wherein the independent parameter includes sheath detection mechanism.

Example 30 is the system of example 16, wherein the first catheter element includes a catheter sheath.

Example 31 is the system of example 16, wherein the second catheter element includes an ablation catheter.

Example 32 is a system for use with an electrophysiological procedure. The system includes a catheter assembly including a plurality of coaxially disposed catheter elements, the plurality of catheter elements including a first catheter element and a second catheter element, the first catheter element forming an elongated lumen defining a longitudinal axis and the second catheter element disposed within the lumen, the first and second catheter elements movable with respect to each other along the longitudinal axis, wherein the first catheter element includes a first tracking sensor configured to generate a first electrical signal and the second catheter element includes a second tracking sensor configured to generate a second electrical signal. A controller is configured to generate a first position of the first catheter element based on the first electrical signal and generate a second position of the second catheter element based on the second electrical signal. In response to a detected longitudinal movement of the second catheter element along the longitudinal axis with respect to the first catheter element based on a sheath detection: apply a historical correction to the first and second positions based on a previously determined first and second position vector to obtain an initially corrected first and second positions, apply a longitudinal adjustment to the initially corrected first and second positions based on a constraint applied to the detected longitudinal movement, and generate an anatomical map of an organ of the electrophysiological procedure with a visualization of the catheter assembly including the longitudinal adjustment.

Example 33 is the system of example 32, wherein the controller is configured to update the previously determined first and second position vector in response to the detected longitudinal movement of the second catheter element along the longitudinal axis with respect to the first catheter element and wherein the updated previously determined first and second position vector is based on a plurality of previously determined first and second position vectors.

Example 34 is a system for use with an electrophysiological procedure. The system includes a catheter assembly including a plurality of coaxially disposed catheter elements, the plurality of catheter elements including a first catheter element and a second catheter element, the first catheter element forming an elongated lumen defining a longitudinal axis and the second catheter element disposed within the lumen, the first and second catheter elements movable with respect to each other along the longitudinal axis, wherein the first catheter element includes a first tracking sensor configured to generate a first electrical signal and the second catheter element includes a second tracking sensor configured to generate a second electrical signal. A controller is configured to track the first catheter element via impedance tracking and to track the second catheter element via magnetic tracking; generate a first position of the first catheter element based on the first electrical signal and generate a second position of the second catheter element based on the second electrical signal. In response to a detected longitudinal movement of the second catheter element along the longitudinal axis with respect to the first catheter element based on a sheath detection: apply a historical correction to the first and second positions based on a previously determined first and second position vector to obtain an initially corrected first and second positions, apply a longitudinal adjustment to the initially corrected first and second positions based on a constraint applied to the detected longitudinal movement, and generate an anatomical map of an organ of the electrophysiological procedure with a visualization of the catheter assembly including the longitudinal adjustment.

Example 35 is the system of example 34, wherein the controller is configured to apply the longitudinal adjustment to the second position with respect to the first position.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an example clinical setting for treating a patient, and for treating a heart of the patient, the example clinical setting having an example electrophysiology system.

FIG. 2 is a block diagram illustrating an example controller for use with the example electrophysiology system of FIG. 1.

FIG. 3 is a diagram illustrating an example process of the example controller of FIG. 2.

FIG. 4 is a schematic diagram illustrating separated example sections of example catheter elements of an example catheter assembly for use with the example electrophysiology system of FIG. 1.

FIG. 5A is a schematic diagram illustrating a context for an implementation of the process of FIG. 3 with the example catheter elements of FIG. 4.

FIG. 5B is a schematic diagram illustrating a representation of device models from tracking the example of catheter elements of FIG. 5A.

FIG. 5C is a schematic diagram illustrating an implementation of a feature of the process of FIG. 3 with the device models of FIG. 5B.

FIG. 5D is a schematic diagram illustrating an implementation of another feature of the process of FIG. 3 with the device models of FIG. 5C.

FIG. 6A-6F are schematic diagrams of the example catheter elements and corresponding device models illustrating a feature of the process of FIG. 3.

FIG. 7 is a block diagram illustrating an example process of a feature of the process of FIG. 3.

FIG. 8 is a schematic diagram of the representation of the device models of FIG. 5B illustrating a concept of the process of FIG. 7.

FIG. 9 is a perspective view of a spatial map further illustrating the concept of FIG. 8.

FIG. 10 is a flow chart illustrating an example feature of the process of FIG. 7.

While the disclosure is amenable to various modifications and alternative forms, specific embodiments have been shown by way of example in the drawings and are described in detail below. The intention, however, is not to limit the disclosure to the particular embodiments described. Rather, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

Examples of electrophysiological procedures and systems in which electroanatomical mapping systems track catheter assemblies are described in this disclosure with electrophysiological testing and ablation systems for illustration. Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a catheter is inserted into the patient and radio frequency waves are transmitted through the catheter to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. Another ablation technique uses electroporation. In electroporation, or electro-permeabilization, an electrical field is applied to cells to increase the permeability of the cell membrane. The electroporation can be reversible or irreversible, depending on the waveform or pulse pattern, strength and duration of the electric field. If the electroporation is reversible, the temporarily increased permeability of the cell membrane can be used to introduce chemicals, drugs, or deoxyribonucleic acid (DNA) into the cell, prior to the cell healing and recovering. Tissue recovery can occur over minutes, hours, or days after the ablation is completed. If the electroporation is irreversible, the affected cells are killed, such as via some form of cell death, such as perhaps programmed cell death through apoptosis for example, or such as traumatic cell death through necrosis for example. Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a relatively safe and effective alternative to the indiscriminate killing of thermal ablation techniques, such as RF ablation and cryoablation. Irreversible electroporation can be used to kill targeted tissue, such as myocardium tissue, by using a selected electric field strength and duration that is effective to kill the targeted tissue but is not effective to permanently damage other cells or tissue, such as non-targeted myocardium tissue, red blood cells, vascular smooth muscle tissue, endothelium tissue, and nerve cells. Irreversible electroporation systems are presented in this disclosure for illustration, but the concepts of catheter and assembly tracking can also apply to other systems.

Cardiac ablation, as well as other electrophysiological procedures, can involve the use of catheter assemblies. A catheter assembly includes a plurality of catheter elements, and catheter elements can include catheters, sheaths, dilators, guidewires, and needles. As electrophysiological procedures move toward less use of fluoroscopy, catheter elements include tracking devices or sensors to facilitate tracking in electroanatomical mapping systems. Typically, catheter elements in a catheter assembly are tracked separately via tracking systems in electroanatomical mapping systems. Because tracking systems are inexact and provide approximations of the positions of the catheter elements within an organ, different catheter elements in a catheter assembly may be rendered in a visualization as displaced or separated from one another even though the catheter assembly incudes one catheter element disposed within another within the patient. It is desirable that the catheter elements be presented in accurate positions with respect to each other and the heart for clinical interpretation. When the tracked elements are physically coaxial, for example, it is desirable that the estimates of the catheter element locations be improved using this information, and that the catheter elements be rendered, such as in a visualization, as coaxial. Similarly, when one of the tracked elements has advanced past another, for example, it is desirable that the catheter elements be rendered as single catheter assembly.

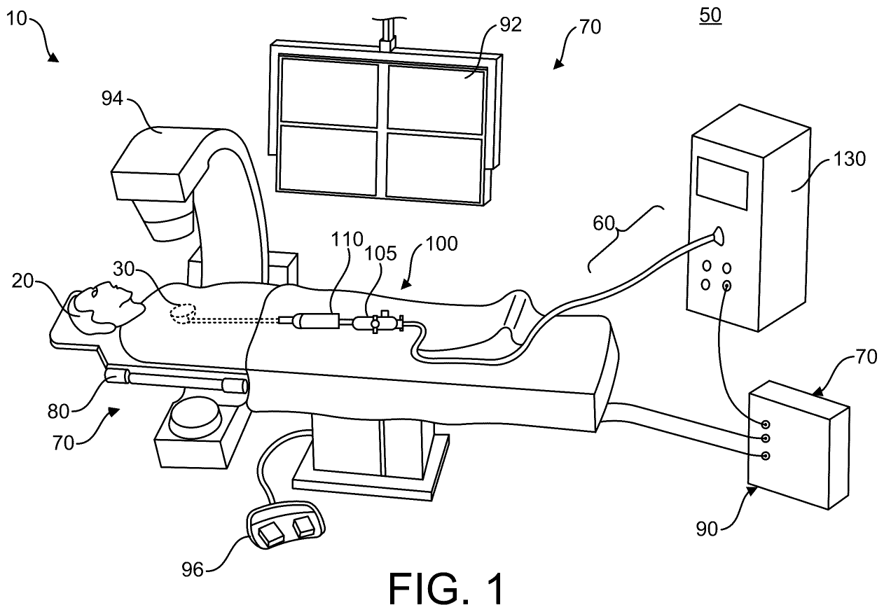

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electroanatomical mapping (EAM) system 70. The example electroporation catheter system 60 includes an elongated catheter assembly 100, which in the example includes an electroporation catheter 105 and an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1

The electroporation catheter system 60 is configured to deliver electric field energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheter elements. For instance, a catheter element such as a sheath or catheter defines a longitudinal axis that passes through a centroid of a cross section of the catheter element, such as the centroid of a cross section of a catheter shaft or a centroid of a cross section of a lumen of a sheath. Coaxial disposed catheter elements include a catheter element disposed within another catheter element such that the longitudinal axes of each catheter element generally follow the same three-dimensional curve or path up to the most distal point that both are present. The catheter elements can include a first catheter element, such as an elongated introducer sheath 110, and a second catheter element, such as an elongated catheter such as electroporation catheter 105. The first catheter element includes an elongated lumen and the second catheter element is disposed within the lumen. In the example, the catheter 105 is disposed within the introducer sheath 110. The first and second catheter elements are movable with respect to each other along the longitudinal axis. For example, a distal end of the catheter 105 can be manipulated to extend from the distal tip of the introducer sheath 110, or the distal tip of the introducer sheath 110 can be retracted from the distal end of the catheter 105. Additionally, the distal end of the catheter 105 can be retracted from the distal tip of the introducer sheath 110. The first catheter element includes a first tracking sensor, and the second catheter element includes a second tracking sensor. For instance, each of the first and second catheter elements can include one or more tracking sensor. Examples of tracking sensors can include magnetic navigation devices and electrodes.

The introducer sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart. In one example, the catheter assembly 100 including the introducer sheath 110 is adapted for use with a transseptal puncture. The left atrium of the heart is a relatively difficult chamber to access percutaneously, and the transseptal puncture permits a direct route to the left atrium via the intra-atrial septum and systemic venous system.

The example catheter 105 includes an elongated catheter shaft and distal end configured to be deployed proximate to the target tissue, such as within a chamber of the patient's heart. The distal end may include a basket, balloon, spline, configured tip, or other electrode deployment mechanism to effect treatment. The electrode deployment mechanism includes an electrode assembly or array. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. In one example, the plurality of electrodes can be formed of a conductive, solid-surface, biocompatible material and are spaced-apart across insulators. Each of the plurality of electrodes is electrically coupled to a corresponding elongated lead conductor that extends along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to a plug in the proximal region of the catheter 105, such as a plug configured to be mechanically and electrically coupled to the electroporation console 130, for example, either directly or via intermediary electrical conductors such as cabling. In one example, the electroporation console 130 is configured to provide an electrical signal, such as a plurality of concurrent or sequential electrical signals, to the electrically connected catheter 105 along lead conductors to the spaced-apart electrodes. In an example of an electroporation catheter, the spaced-apart electrodes are configured to generate a selected electrical field proximate the target tissue, based on the electrical signals from the electroporation console 130, to effect electroporation.

A selected electrical field can be generated with the electrodes to effect electroporation. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

The electroporation console 130 is configured to control aspects of the electroporation catheter system 60. In embodiments, the electroporation console 130 is configured to provide one or more of the following: modeling the electric fields that can be generated by the electroporation catheter 105, which often includes consideration of the physical characteristics of the electroporation catheter 105 including the electrodes and spatial relationships of the electrodes on the electroporation catheter 105 and whether the electroporation catheter 105 is in bipolar or monopolar mode; generating the graphical representations of the electric fields, which often includes consideration of the position of the electroporation catheter 105 in the patient 20 and characteristics of the surrounding tissue; and overlaying, on the display 92, the generated graphical representations on an anatomical map. In some examples, the electroporation control console 130 is configured to generate the anatomical map. In some examples, the EAM system 70 is configured to generate the anatomical map for display on the display 92.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the OPAL™ HDx mapping system (formerly available under the trade designation RHYTHMIA™ HDx mapping system) marketed by Boston Scientific Corporation. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume. In general, impedance tracking systems employ a field map or other mechanism to relate localization fields to spatial positions within the body. When the electrode sensors on the impedance tracked device measure the localization fields during tracking, the impedance tracking system estimates the spatial location of the electrodes. In some systems, the field map is computed using, for example, mathematical models of the electric fields in the body induced by the surface electrodes. In other systems, this field map is measured using a magnetically tracked device that measures the electric fields at points in space known from magnetic information. Other possibilities are envisioned, for example, the field map could be stored on the computer system and read by the impedance tracking system. In one example, assembly tracking addresses making consistent two different tracking methodologies. The illustrated example relates to magnetic and impedance tracking methodologies. In general, however, whenever two (or more) sources of location information are employed and one source of location information is more accurate than the other, the more accurate source of location information, and knowledge of the physical relationship of both together, are used to improve the less accurate one.

The EAM system 70 can be equipped for both magnetic and impedance tracking capabilities. In such examples, impedance tracking is made possible by first creating a map of the electric fields induced by the electric field generator within the cardiac chamber of interest using a probe equipped with a magnetic location sensor, as is possible using the OPAL HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

An example EAM system 70 tracks locations of catheter elements of a catheter assembly 100 using processes known in the art. In one example of an EAM system 70 provides equations that include the position and perhaps velocity of each of the electrodes. Tracked devices, such as catheter elements of the catheter assembly 100, are characterized in the EAM system 70 as device models that include electrode locations and tangents among other parameters. In one example presented for illustration, tracking can include the application of external electrodes that generate a plurality of non-parallel electric fields throughout the body for impedance tracking. A magnetically tracked device is used to associate those electric field values, or impedance values, with each position in space as determined magnetically. A resulting data structure is presented as a field map. In the case of only an impedance device being tracked, the field map is inverted to estimate the location of each electrode. The estimated locations are smoothed, and the processes include a variety of constraints on the shape of the flexible device comprised of all its electrodes taken together. The body is nearly transparent to magnetic fields, but the geometry and electrical properties of body tissues affect the magnitude and shape of the electric fields dramatically, impedance tracking is less accurate than magnetic tracking.

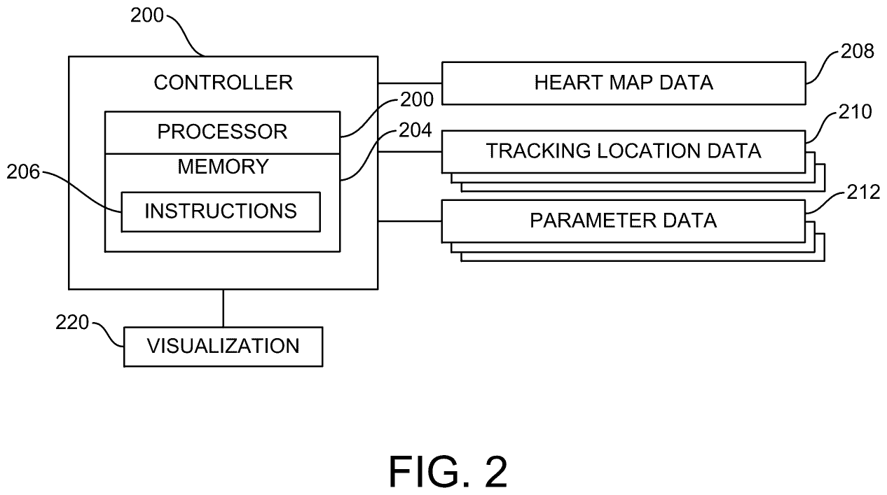

FIG. 2 illustrates an example controller 200 that can be used with the example electrophysiology system 50, such as a controller of the example electroporation catheter system 60, which may include a controller of the example EAM system 70 such as the mapping and navigation controller 90. The controller 200 can be implemented to provide a device model and visualization of a catheter assembly as a single unit within a patient even when the catheter elements of the catheter assembly are independently tracked and independently tracked with different methodologies during an electrophysiological procedure. The controller 200 can include a processor 202 and a memory 204. The memory 204 stores processor executable instructions 206. In one example, the processor executable instructions can be in the form of a program, such as a computer program or application. The processor 202 can execute the instructions 206 that can be included in configuring the controller 200. In one example, the controller 200 can be implemented to include a computing device such as a laptop computer, a workstation, a desktop computer, a tablet, or a smartphone. In such examples, the controller 200 can include additional components such as a display, a touchscreen, speakers or other output devices, a keyboard or other input devices, or communication circuitry such as computer network adapters. The controller 200 may be implemented in a variety of architectures and components, such as the processor 202 and memory 204, may be distributed in various locations.

In one example, the processor 202 may include a plurality of main processing cores to run an operating system and perform general-purpose tasks on an integrated circuit. The processor 202 may also include built-in logic or a programmable functional unit, also on the same integrated circuit with a heterogeneous instruction-set architecture. In additional to multiple general-purpose, main processing cores and the application processing unit, controller 200 can include other devices or circuits such as graphics processing units or neural network processing units, which may include heterogeneous or homogenous instruction set architectures with the main processing cores. For example, the controller 200 may be used to perform other tasks such as in the case of a computing device including the resonance sound amplification device.

Memory 204 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 202. Any such computer storage media may be part of the controller 200 and implemented as memory 204. Memory 204 is a non-transitory, processor readable memory device. Accordingly, a propagating signal by itself does not qualify as storage media or memory 204.

The controller 200 may be configured to receive inputs or information from the electrophysiology system 50, such as inputs from the electroporation catheter system 60 and EAM system 70 including the electroporation console 130 and the mapping and navigation controller 90, for storage in memory 204 and use by the instructions 206. For example, the controller 200 can receive an input representative of the anatomical map of the heart, or heart map data 208, which heart map data 208 can include the data regarding representations of the geometric anatomical map of the heart and the electro-anatomical map of the heart, such as from the EAM system 70. Additionally, heart map data 208 can include annotations, markings, or user-added tags of the heart that may include markings of anatomical locations of interest or other data to generate visualizations a clinician may find of interest during a procedure. The controller 200 can also receive tracking location data 210 for each catheter element of the catheter assembly. The tracking location data can be generated by a magnetic tracking, impedance tracking, or other tracking techniques, and can be generated via the EAM system 70 or other features of electrophysiology system 50. Tracking location data can be received periodically, such as at a sampling rate. In one example, the sample rate for tracking location data 210 can be 20 Hertz. In one example, tracking location data 210 includes information used to determine the position of sensors on the catheter elements with respect to the heart. Also, tracking location data 210 can include information regarding how many electrodes in the catheter are exposed from underneath the sheath, such as sheath detection data if the electrophysiology system 50 includes sheath detection mechanisms. In some examples, the processor 200 can receive parameter data 212 including catheter assembly parameter data and patient physiological parameter data. Catheter assembly parameter data of parameter data 212 can include information regarding the parameters of the catheter elements such as number and spacing of electrodes on catheter element, the type of tracking methodology used, and various other parameters such as mechanical properties and state, such as, in one embodiment, properties of the catheter elements that may be used to determine constraints. In one catheter assembly parameter data of parameter data 212 can include a separate input for each catheter element. Patient physiological parameter data of parameter data 212 can include information as stage of the cardiac cycle, stage of respiratory cycle, temperature data, location of anatomy, elapsed time of procedure, and other information associated with the received tracking location data 210, such as parameters associated with each received data sample. Other parameter data can include environmental data, such as the type and amount of ambient electromagnetic noise present in the environment that impacts the accuracy of magnetic or impedance tracking fields and subsequently impacting localization accuracy. In one example, heart map data 208, tracking location data 210, and parameter data 212 are stored in memory 204 for use by the processor 202 executing the instructions 206.

The controller 200 is configured to generate a visualization 220 that can include determined location of the catheter assembly as a single unit based on the received tracking data of the catheter elements with reference to the anatomical map of the heart. In one example, the controller 200 is configured to generate a visualization 220 based on constraints applied to the tracking data 210 and catheter parameter data 212 with heart map data 208.

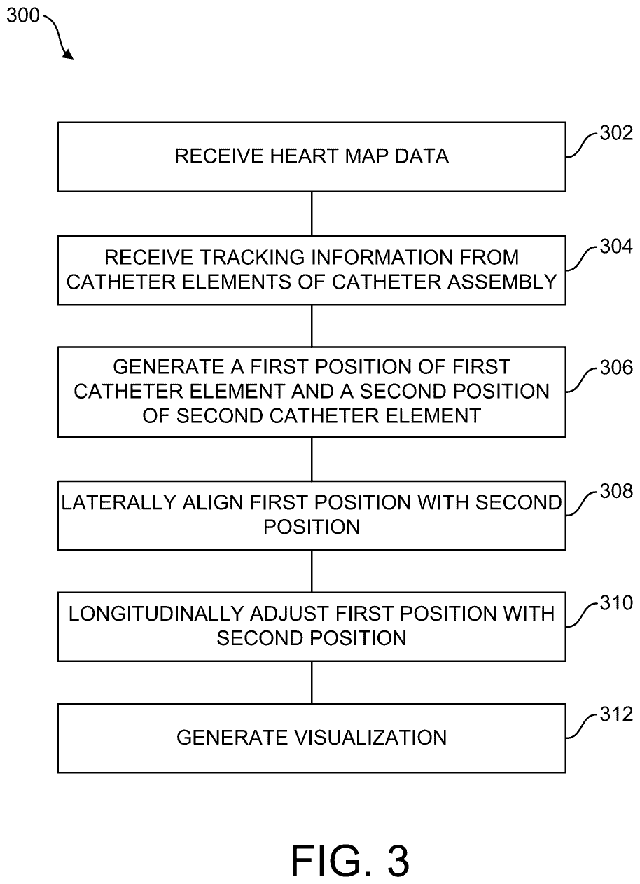

FIG. 3 illustrates a process 300 of configuring a controller, such as controller 200, while tracking a catheter assembly having plurality of catheter elements, catheter element each having a tracking sensor. In the example process 300, the catheter assembly includes a plurality of coaxially disposed catheter elements, and the catheter elements include a first catheter element and a second catheter element. In the example, the first catheter element forms an elongated lumen, and the second catheter element is coaxially disposed within the lumen. The first and second catheter elements are movable with respect to each other along an axis. Examples of first catheter element can be a sheath or a dilator/sheath assembly, and an example of second catheter element can be an ablation catheter disposed within a lumen of the sheath. The first catheter element includes a first tracking sensor, and the second catheter element includes a second tracking sensor. Process 300 can receive parameters regarding the first catheter element and second catheter elements via catheter parameter data of parameter data 212.

In one example, process 300 can be implemented as set of processor-executable instructions, such as instructions 206, stored in a non-transitory memory, such as memory 204 to be executed by a processor 202 to configure controller 200. The instructions to implement process 300 can be configured to receive information, such as to retrieve from memory 204, heart map data 208 and tracking location data 210 and parameter data 212. Further, the instructions to implement process 300 can be configured to write to heart map data 208 and to generate a visualization, such as visualization 220 on a display of graphical representations.

Process 300 includes configuring the controller 200 to receive electroanatomical map data of an organ, such as receive heart map data 208, at 302. The controller 200 is configured to track a catheter assembly within the heart based on a first electrical signal from the first tracking sensor and based on a second electrical signal from the second tracking sensor at 304. In the example, the positions of the catheter elements are tracked generally concurrently. The controller 200 is configured to generate a first position of the first catheter element based on the first electrical signal and to generate a second position of the second catheter element based on the second electrical signal at 306. For instance, the controller 200 is configured to generate the position of the magnetic tracking sensor or each electrode on the first catheter element based on its set of magnetic or electrical signals, and similarly for the second catheter element. The first and second catheter element positions can each include a set of data determined by the mapping and navigation controller 90 regarding the catheter element in space, such as the catheter pose or the three-dimensional curve of the distal portion of the catheter having the tracking sensor as included in tracking location data 210. In one example, the first catheter element position includes a first location in space and a first tangent of the catheter element as determined by the EAM system 70, and the second catheter element position includes a second location in space and a second tangent of the catheter element as determined by the EAM system 70. In an example as used in this disclosure, two objects are considered coaxial at a point if the objects touch at the point and have a same tangent at the point. The first position is laterally aligned with the second position based on a lateral displacement of the first catheter element relative to the second catheter element at 308. For example, the first location and the first tangent are laterally aligned with the second location. and the second tangent based on a lateral displacement and a rotational displacement determined from the first location and first tangent and the second location and second tangent, respectively. For example, the tangents at the point can be aligned by a rigid body transformation such as rotation or by using points along one device to modify locally the tangent of the other device. The first position is longitudinally adjusted with respect to the second position based on a detection of the second tracking sensor using the first tracking sensor at 310. For example, the first position is longitudinally adjusted with respect to the second position based on a detection of the first tracking sensor of the second tracking sensor. The controller is configured to generate an electroanatomical map of an organ of the electrophysiological procedure with a visualization of the catheter assembly having a device model of the first catheter element constrained with a device model of the second catheter element at 312.

Lateral alignment at 308 and longitudinal adjustment at 310 can be performed based on various determinations from the tracking location data 210 and catheter parameter data of parameter data 212. Lateral alignment at 308 can be applied to correct departure or displacement in the device models of the catheter elements. In one example, departure or displacement is addressed via translation, or moving one device along a vector from one point to another. Further, lateral alignment at 308 can be applied to correct deflection, or rotation of the device models of the catheter elements. In one example, rotation is applied by moving one device by an angle. Longitudinal adjustment at 310 can be applied to correct protrusion, or longitudinal displacement of one catheter element with respect to the other catheter element. A determination of the number of electrodes exposed or uncovered by the sheath can be used to inform the longitudinal adjustment at 310. In one example of a tracked catheter and sheath, sheath detection—or the technology used to determine whether and how many electrodes of the catheter are exposed from under the sheath—is used to place constraints on the relative locations of the device models of the catheter and sheath, such as for longitudinal adjustment at 310. Rotational alignment at can be applied to align the local tangents by rotating one or both objects or by using one object to modify the local shape of the other object. In another example of lateral alignment at 308, the positions and poses of the catheter elements can be determined by tracking the catheter elements of the catheter assembly, applying a likely curve of the catheter assembly using reduced bending energy as determined from catheter parameter data of parameter data 212, and finding the positions and poses again but constrained to the likely curve. This example process may be repeated.

One or more catheter of the devices models of the catheter elements can be aligned and adjusted. The determinations of lateral alignment at 308 and longitudinal adjustment at 310 can assign weights to each of the device models of the catheter elements based on the confidence that the tracked catheter element is in the site in space with respect to the heart as represented in the tracking. In general, the confidence in the tracking of the two elements can be used to weight the operations applied to each element to affect the desired constraints. For example, magnetic tracking is relatively accurate and closely represents the location of the catheter element site in space with respect to the heart. In the case of a catheter configured for magnetic tracking and the sheath configured for impedance tracking, an approach is to give device model of the catheter a high confidence value (e.g., 1.0 on a scale of 0.0 to 1.0) and the device model of the sheath is laterally aligned with the tracked position of the device model of the catheter given the relative accuracy of magnetic tracking. In the case of a catheter configured for impedance tracking and the sheath configured for impedance tracking, the device model of the catheter can be given a confidence value of less than 1.0 and the device model of the sheath is given a confidence value of less than 1.0 as based on information from the catheter parameter data regarding the likelihood of accurate tracking. In this case, the location of both the catheter and sheath is adjusted based upon their relative weights. Alternatively, the user may select to control these adjustments manually, for example, to adjust the sheath and not the catheter.

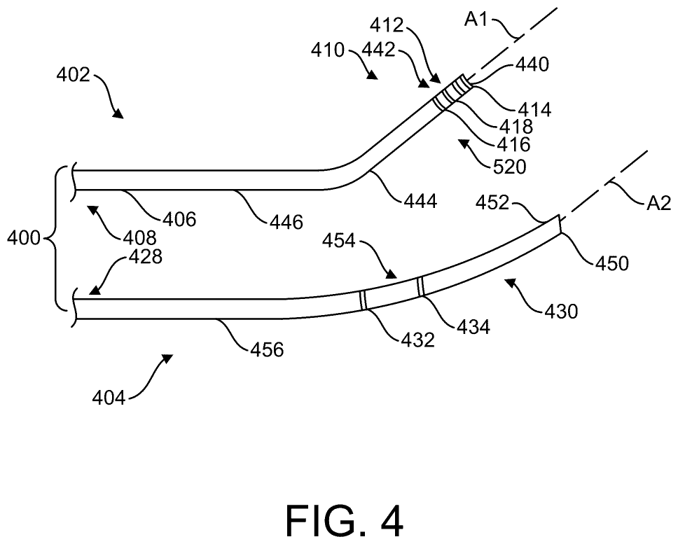

FIG. 4 illustrates an example catheter assembly 400 having a plurality of separate distal sections of catheter elements including a catheter 402 and a sheath 404, which can be an example of catheter 105 and sheath 110 of FIG. 1. Distal section of catheter 402 includes a shaft 406 disposed on a longitudinal axis A1 and having a proximal region 408 and a distal region 410. The distal region 410 in the example catheter 402 includes a plurality of electrodes 412 for ablating tissue. The plurality of electrodes 412 in the example includes a tip electrode 414 and ring electrodes 416, 418 carried on a distal portion 420 of the shaft 406. The catheter shaft 406 can be flexible along its entire length of the distal section, but in the example shown, the distal portion 420 carrying the electrodes 412 is rigid and not bendable. The catheter shaft 406 can include a navigation device (not shown) in or near the distal portion 420 within the shaft 406 for magnetic tracking.

The distal section of the sheath 404 can define a lumen along longitudinal axis A2 such that the catheter 402 can be carried inside the lumen. The distal section of the sheath 404 includes a proximal region 428 and a distal region 430. The distal region 430 includes a plurality of ring electrodes 432, 434 for impedance tracking as well as sheath detection of catheter electrodes 412. In one example, the electrophysiology system 50 applies sheath detection to determine whether the catheter distal portion 420 is disposed within the sheath 404 and/or the number of catheter electrodes 412 that are exposed from underneath the sheath 404. An example of sheath detection is described in U.S. patent application Ser. No. 16/686,591, titled SHEATH DETECTION USING LOCAL IMPEDANCE INFORMATION, filed Nov. 18, 2019, to Salehi et al., and assigned to the present assignee, the contents of which are incorporated by reference into this disclosure to the extent it is consistent with this disclosure. In this example, the distal section of the sheath 404 is flexible along its entire length including along the sheath distal region 430 with the exception, possibly, of a relatively small distal-most portion.

The EAM system 70 generates device models of the distal sections of the catheter elements 402, 404 for processing. In one example, the device models include data from the distal tip of the catheter element to a proximal bound of an articulable segment of the catheter element, or distal section. For example, a device model for each catheter element 402, 404 includes a point and three device segments. The point is a tip of the catheter element. The distal-most point of the catheter element is the tip, such as catheter tip 440 and sheath tip 450. The device segments of the device model of the catheter element include a head segment, a neck segment, and a body segment. The head segment 442, 452 includes the distal-most rigid tip segments of the catheter element. The neck segment 444, 454 includes the flexible articulable segment proximal to the head segment 442, 452, respectively. The body segment 446, 456 includes the flexible segment most proximal on the catheter element, which can be flexible but passively not articulable.

Based on values of the electrode locations and electrode tangents, the EAM system can constrain each device segment of the device model into a geometric shape. The geometric shapes used to represent the device segments can include a point, line, circle, any other defined geometric shape, or a spline. For a catheter 402, an example device model fits as follows: the head segment 442 is fit as line to the electrodes 412, the tip 440 is fit as a point at the end of the head segment 442, and the neck segment 444 is fit as spline if, the neck of the catheter 402 is extended from the sheath 404. For a sheath 404, an example device model fits as follows: the neck segment 454 is fit as a spline (in other examples, the segment can be fit as a circle or line), the head segment 452 is fit as a line using the tangent of the distal most electrode 432, and the tip 450 is fit as a point at the end of the head segment 452. For a flexible device such as the sheath, a spline fit to the electrodes can take the shortest path that connects the electrodes subject to smooth constraints such as continuity of the first and second spatial derivatives. This path, however, may not satisfy the known distance between electrodes for example. To meet this constraint, a simple geometric object such as a circle can be used to constrain the spline over some or all of the flexible device segment. In one example, a geometric object such as a circle may be used to add additional points that constrain a spline to be fit using only points. In another example, a geometric object such as a circle may be used to compute tangents that constrain a spline to be fit using both points and tangents. In another example, a geometric object such as a circle may be used to compute tangents and distances between electrodes that constrain a spline to be fit using points, tangents and distanced between electrodes.

In the following examples, the catheter 402 is tracked via magnetic tracking and the sheath 404 is tracked via impedance tracking, and the tracking of the catheter is given a confidence value of 1.0 such as on a scale of 0.0 to 1.0, so the during the application of process 300, the device model of the sheath 404 is adjusted and aligned to fit the model of the catheter 402. In another embodiment the tracking of the sheath is given a confidence value of 1.0, so the device model of the catheter is adjusted to align and fit the model of the sheath. In another embodiment both devices are given confidence values between 0.0 and 1.0, due to noise levels or other metrics of tracking accuracy, so the two device models are shifted toward each other, e.g., toward some noise-weighted average. Also, the catheter assembly 400 implements sheath detection. In an embodiment, the process 300 receives inputs regarding the positions of the catheter elements and implements the constraints periodically, such as several times a second. For example, the process can include a sampling rate of 20 Hertz. Other sampling rates are contemplated.

Sheath detection data can be included with catheter location data 210 as including a state or a number of exposed electrodes on the catheter 402. For instance, sheath detection data can report a covered state, a partially covered state, and an uncovered state. A covered state is when the sheath 404 entirely covers a catheter 402, such as when the sheath 404 covers all the electrodes 412 on the catheter 402. A partially covered state is when some, but not all, of the electrodes 412 on a catheter 402 are exposed from underneath the sheath 404. An uncovered state is when all the electrodes 412 of the catheter 402 are exposed from underneath the sheath 404. In other embodiments, the coverage state may be determined by purely geometric means that quantify the geometric relations between the two device models. In other cases, the coverage state may be determined initially by input from the users, and state transitions may be achieved by a state machine that uses geometric information to control transitions between neighboring states.

In a covered state, constraints are applied to the device models of the catheter elements 402, 404 if the catheter tip 440 is advanced beyond (distal) the most proximal sheath electrode 432. A point on the sheath body 456 closest to the tip of the catheter head 442 is determined. The device model of the sheath body 456 is translated to be coaxial with the nearest point of the device model of the catheter head 442. In terms of coaxial in space, the two device models are constrained to intersect at that point and have parallel tangents at that point. Also, the sheath tip 450 is adjusted to be consistent with the number of exposed electrodes 412, which is zero in the covered case. To translate the device model of the sheath 404 to the device model of the catheter 402 in the covered state, which includes a transformation of one device along a vector from one point to another point, the device model of the sheath body 456 is moved laterally to the device model of the catheter head 442 and is moved longitudinally to satisfy the sheath detection of no exposed electrodes. The device model of the sheath 404 is also rotated or otherwise modified about the tip of the device model of the catheter to match local tangents of the catheter 402.

In a partially covered state, the device model of the sheath head 452 is translated to be coaxial with the device model of the catheter head 442, and the device model of the sheath tip 450 is translated to expose the number of electrodes indicated by the sheath detection data. To translate the device model of the sheath 404 to the device model of the catheter 404 in the partially covered state, which includes a transformation of a vector from one point to another point, the shortest vector between the device models of the sheath tip 450 and catheter head 442 defines a translation. The device model of the sheath 404 is moved longitudinally to satisfy the sheath detection data of the proper number of exposed electrodes. The device model of the sheath tip 450 is also rotated or otherwise modified about the device model of the catheter tip 440 to match local tangents of the catheter tip 440.

In an uncovered state, constraints are applied to the device models of the catheter elements 402, 404 if the catheter tip 440 protrusion is less than a bound of the catheter neck 444. The device model of the sheath head 452 is translated to be consistent with the articulation of the device model of the catheter neck 444. Also, the device model of the sheath tip 450 is longitudinally adjusted to be consistent with the number of exposed electrodes 412, which is all the electrodes in the uncovered case. To translate the device models of the sheath 404 to the catheter 402 in the covered state, which includes a transformation of the one device along a vector from one point to another point, the device model of the sheath tip 450 moved laterally to the device model of the catheter head 442, and the device model of the sheath tip 450 is moved longitudinally to satisfy the sheath detection of all exposed electrodes. The device model of the sheath tip 450 is also rotated or otherwise modified about the device model of the catheter tip 440 to match local tangents.

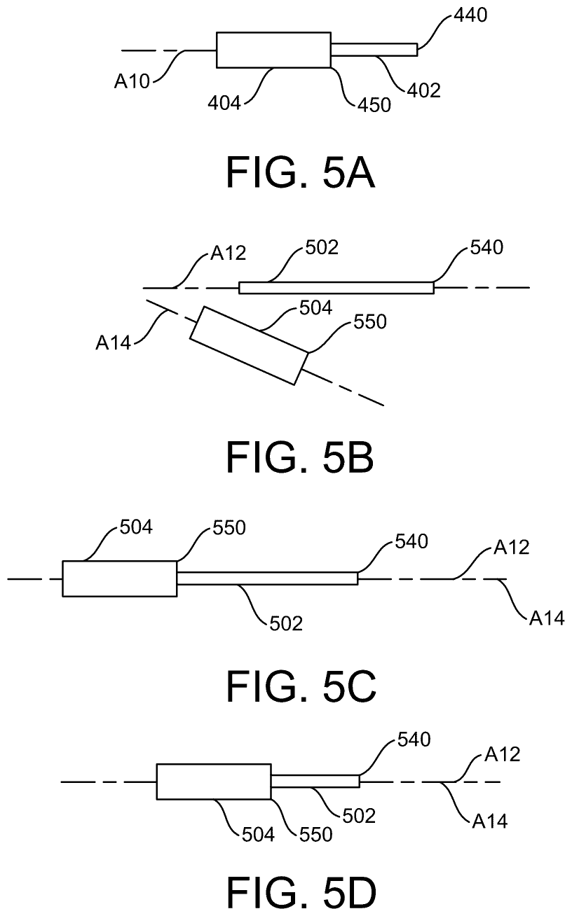

FIG. 5A-5D illustrate an example implementation of the process 300 as applied to catheter assembly 400. In the illustrated example implementation, the position of the catheter 402 is determined via magnetic tracking and the position of the sheath 404 is determined via impedance tracking. In this case, the tracking of the catheter 402 is given a confidence value of 1.0, so the during the application of process 300, the device model of the sheath 404 is adjusted and aligned to fit the determined position of the device model of the catheter 402. Also, the electrophysiology system 50 includes sheath detection technology and the process 300 implements use of sheath detection data as part of catheter location data 210.

FIG. 5A illustrates the actual location of the catheter assembly 400 in space and deployed within a heart chamber. The catheter assembly 400 includes coaxially disposed catheter elements such as the catheter 402 and sheath 404. The sheath 504 forms an elongated lumen and the catheter 402 is disposed within the lumen. The catheter 402 and sheath 404 are movable together in space and with respect to each other along an axis A10, and, as illustrated, the catheter 402 extends from sheath 404 such that the catheter tip 440 is extended past the sheath tip 450.

FIG. 5B illustrates a visual representation of the device model catheter assembly 500 constructed because of the independent tracking of the catheter elements 402, 404 prior to application of the process 300. The device model of the catheter assembly 500 includes a device model of the catheter 502 and a device model of the sheath 504 corresponding with the catheter 402 and sheath 404, respectively. FIG. 5B is an exaggerated representation of the divergence of the device models of the catheter elements 502, 504 for illustration. The device model of the catheter 502 includes a device model catheter tip 540, and the device model of the sheath 504 includes a device model sheath tip 550. As illustrated, the device model of the catheter 502 is laterally displaced from the device model of the sheath 504 in departure prior to the application of process 300. Also as illustrated, the device model of the catheter 502 is disposed along axis A12 and the device model of catheter 504 is disposed along axis A14, in which axes A12 and A14 are not coincident but are deflected via rotational displacement prior to the application of process 300. Despite that the catheter assembly 400 is coaxial within the heart, the device models of the catheter 502 and sheath 504 appear to float and appear to be in different positions in space according to the independent tracking. The process 300 will laterally align the device model of the sheath 504 with the device model of the catheter 502 at the position of the device model of the catheter 502. For instance, the axes A12, A14 of the device models 502, 504 in the illustration will be made parallel and then overlapping. Process 300 will longitudinally adjust the align the device model of the sheath 504 with the device model of the catheter 502 at the position of the device model of the catheter 502.

FIG. 5C illustrates a visual representation of the device model catheter 502 and device model sheath 504 laterally aligned in process 300, such as at 308. In the example, the position of the device model of the sheath 504 is translated to the position of the device model of the catheter 502 to correct departure. Additionally, the device model of the sheath 504 is rotated to the position of the catheter 502 to correct deflection. In the example, axes A12 and A14 are now coincident to intersect and include parallel tangents. Also as illustrated, the device model of the catheter 502 protrudes, or is still longitudinally or axially displaced, from the device model of the sheath 504.

FIG. 5D illustrates a visual representation of the device model catheter 502 and device model sheath 504 longitudinally adjusted in process 300, such as at 310. In the example, the position of the device model of the sheath 504 is translated on axis A14 to the position of the device model of the catheter 502 to correct protrusion, which may be based on sheath detection data, such as on the number of electrodes 412 exposed on the catheter 402. For example, as the catheter 402 is extended from the sheath 404 to expose three electrodes 412, as indicated in FIG. 5A, the device model of the sheath 504 is longitudinally adjusted along the axis to indicate three device model electrodes 512 exposed on the device model of the catheter 502. As indicated, axes A12 and 14 are coincident. Departure, deflection, and protrusion are corrected as illustrated in FIG. 5D, and the device model of the catheter assembly 500 can be provided for presentation in a visualization at 312.

Process 300 corrects the device models for tracked catheter elements using translation, rotation, or shape deformation computed independently for each data sample, such as several times a second. Using sheath detection on spaced-apart electrodes 412, however, can cause the visualization of the device models of the catheter assembly 500 to appear to jump from electrode to electrode when the physical movement of the catheter 402 is, in fact, smoothly moving with respect to the sheath 404 within the patient. For instance, in an example in which the corrections to the position of the sheath 404 are computed as the minimal correction to satisfy the applied constraints, the corrected sheath location (with respect to the catheter location) remains at the distal edge of the first exposed electrode even when the catheter 402 is further advanced toward exposing the second electrode from under the sheath 404. Once sheath detection determines the catheter 402 has been advance from the sheath 404 to expose the second electrode, the device model of the sheath 504 will jump in the visualization to illustrate two exposed electrodes 512.

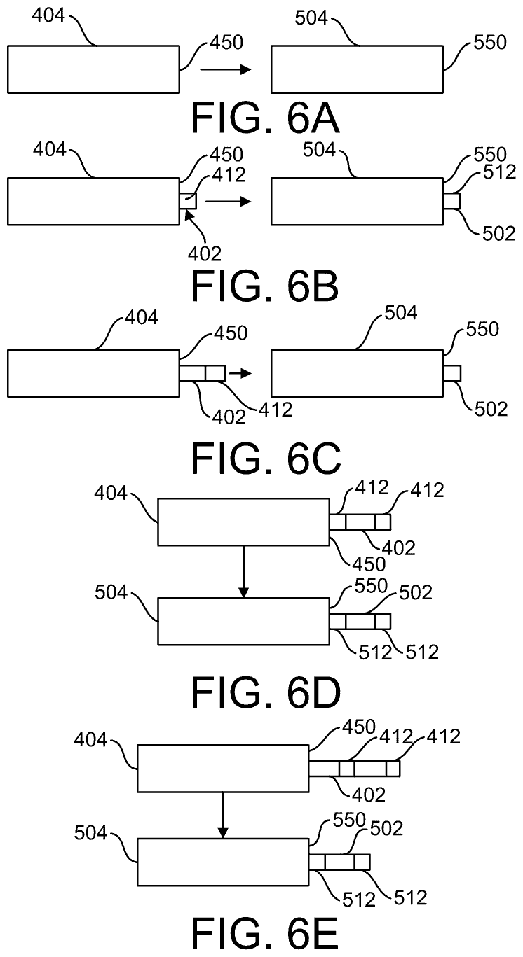

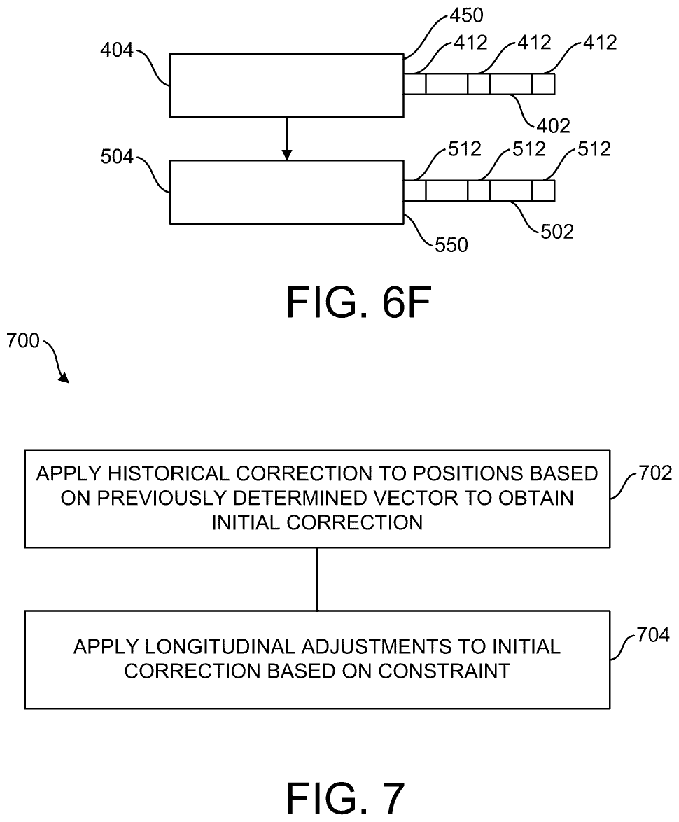

This effect is illustrated in FIGS. 6A-6F. The physical catheter assembly 400 includes a catheter 402 having at least three electrodes 412 and a sheath. The process 300 applies sheath detection to the device model of the catheter assembly 500. The catheter 402 is gradually advanced from the sheath tip 450 from FIGS. 6A to 6F, and the resulting associated visualizations are illustrated in each figure.

In FIG. 6A, the sheath 404 covers the catheter 402 and the electrodes, and the associated visualization 601 illustrates the device model of the sheath 504 covering the device model of the catheter 502.

In FIG. 6B, one electrode 412 on the catheter 402 is exposed from underneath the sheath 404, and the associated visualization 602 illustrates the device model of the catheter 502 with one electrode 512 at the device model tip of the sheath 550.

In FIG. 6C, the catheter 402 has extended further from the sheath tip 450 from the position illustrated in FIG. 6B, but because only one electrode 412 is exposed, the associated visualization 603 illustrates the device model of the catheter 502 with one electrode 512 at the device model tip of the sheath 550.

In FIG. 6D, a second electrode 412 on the catheter 402 is exposed from underneath the sheath 404, and the associated visualization 604 illustrates the device model of the catheter 502 with two electrodes 512 at the device model tip of the sheath 550. In a real-time visualization, the catheter 502 appears to jump from one exposed electrode 512 to two exposed electrodes 512.

In FIG. 6E, the catheter 402 has extended further from the sheath tip 450 from the position illustrated in FIG. 6D, but because only two electrodes 412 are exposed, the associated visualization 605 illustrates the device model of the catheter 502 with two electrodes 512 at the device model tip of the sheath 550.

In FIG. 6F, a third electrode 412 on the catheter 402 is exposed from underneath the sheath 404, and the associated visualization 606 illustrates the device model of the catheter 502 with three electrodes 512 at the device model tip of the sheath 550. In a real-time visualization, the catheter 502 appears to jump from two exposed electrode 512 to three exposed electrodes 512.

User experience is improved if the visualization at 312 of the device models of the catheter assembly 500 are constrained to represent the actual physical catheter assembly 400 and to present movement of the device models of the catheter elements smoothly and representative of physical movements.

FIG. 7 illustrates a process 700 implemented to longitudinally adjust the first position of a first catheter element with a second position of a second catheter element at 310 of process 300. Process 700 can be applied to a catheter assembly including coaxially disposed catheter elements, such as a first catheter element and a second catheter element. The first catheter element, such as a sheath, forms an elongated lumen defining a longitudinal axis and the second catheter element, such as an ablation catheter, is disposed within the lumen. The first and second catheter elements are movable with respect to each other along the longitudinal axis. The first catheter element includes a first tracking sensor configured to generate a first electrical signal and the second catheter element includes a second tracking sensor configured to generate a second electrical signal. The tracking sensors can include tracking electrodes or a combination of magnetic tracking sensors and tracking electrodes for exposure relative to the sheath, such as via a sheath detection mechanism. A controller, such as controller 200, is configured to generate a first position of the first catheter element based on the first electrical signal and generate a second position of the second catheter element based on the second electrical signal at 702. In response to a detected longitudinal movement of the second catheter element along the longitudinal axis with respect to the first catheter element, the controller applies a historical correction to the first and second positions based on a previously determined first and second position vector to obtain an initially corrected first and second positions at 704. In response to a detected longitudinal movement of the second catheter element along the longitudinal axis with respect to the first catheter element, the controller applies a longitudinal adjustment to the initially corrected first and second positions based on a constraint applied to the detected longitudinal movement at 706. As part of generating a visualization at 312, the controller generates an anatomical map of an organ of the electrophysiological procedure with a visualization of the catheter assembly including the longitudinal adjustment at 708.

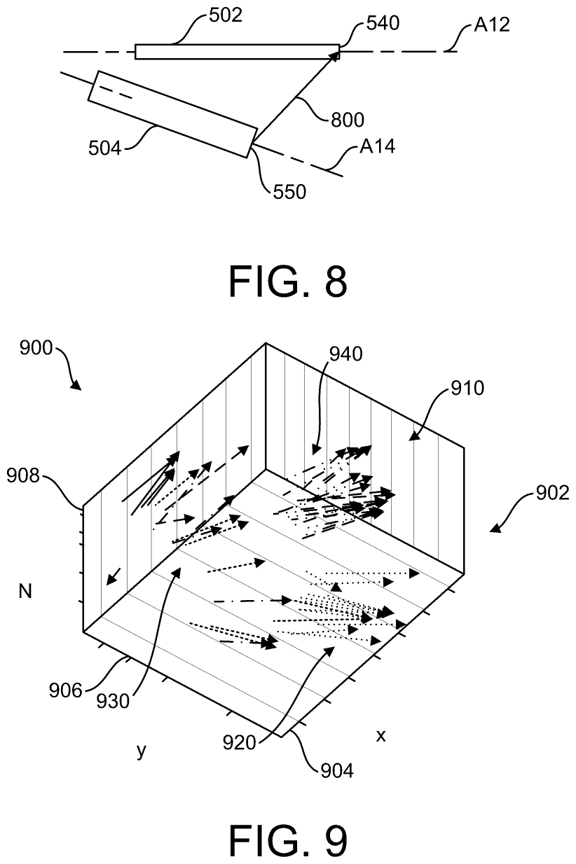

FIG. 8 illustrates a representation of a first and second position vector, or correction vector 800 in connection with the representation of the device model catheter assembly 500 constructed because of the independent tracking of the catheter elements 402, 404 prior to application of the process 300 of FIG. 5B. As described, the physical catheter assembly 400 is indicated in FIG. 5A, and the FIGS. 5B and 8 illustrate the device model of the catheter 502 is laterally displaced from the device model of the sheath 504 in departure prior to the application of process 300. The correction vector 800 in the example is in three-dimensional space and extends from the tip of the device model of the sheath 550 in the three-dimensional space to the tip of the device model of the catheter 540 in the three-dimensional space. In one example, the correction vector 800 can be stored in the memory 204 of the controller as sets of coordinates of a previously determined correction vector 800. Other data can be stored with the correction vector 800, such as a parameter of the electrophysiological procedure. For instance, the correction vector 800 can be included with a time or time elapsed in the electrophysiological procedure, a location with respect to anatomy, or anatomical location of the correction vector 800, an associated period of the respiration cycle of the correction vector 800, a temperature, and other parameters. In one example, a correction vector 800 can be generated with the controller 200 and stored into memory 204 for each instance of a detected longitudinal movement of the sheath 404 with respect to the catheter 402 over the course of an electrophysiological procedure. For instance, a correction vector 800 can be generated and stored for each instance that an independent parameter related to electrodes exposure relative to a sheath, such as sheath detection, determines the sheath 404 exposes or covers an electrode 412 on the catheter 402 over the course of an electrophysiological procedure.

FIG. 9 illustrates a correction map 900 of representations of all correction vectors 910 in a three-dimensional space 902 over a course of an electrophysiological procedure in which the sheath is corrected to match the catheter. For instance, the correction vectors 910 can represent all previously determined correction vectors of the electrophysiological procedure. The three-dimensional-space is indicated in the example via cartesian coordinates 904, 906, 908 with respect to a reference of the EAM system 50. For example, the correction map 900 can be overlaid onto an anatomical map of the heart, and each correction vector 800 of the plurality of correction vectors 910 can correspond with a location of the heart where the sheath was corrected to align with the catheter both laterally and longitudinally with high confidence. Accordingly, a subset of correction vectors can be determined based on anatomical location of the catheter assembly 400. When each correction vector 800 is associated with parameters of the electrophysiological procedure, such as time or time elapsed, subsets of the correction vectors can be determined based on the physiological parameters. In the illustrated example, a first subset of the correction vectors 920 are based on first time period, such as more recent time period in the electrophysiological procedure; a second subset of the correction vectors 930 are based on a second time period, such as a most distant time period in the electrophysiological procedure; and a third subset of the correction vectors 940 are based on a third time period, such as an intermediate time period in the electrophysiological procedure.

The controller applies a historical correction to the first and second positions based on a previously determined first and second position vector to obtain an initially corrected first and second positions at 704. In one example, the previously determined first and second position vector is based on single correction vector 800 of the plurality of correction vectors 910. For instance, the single correction vector on which the previously determined first and second position vector is based can be the first in time stored correction vector or the most recent in time stored correction vector. That correction vector is applied to determine the historical correction to the first and second positions. In another example, the previously determined first and second position vector is based on an amalgamation of correction vectors 800 of the plurality of correction vectors 910. For instance, the amalgamation of correction vectors 800 can be based on all of the correction vectors 910 in the correction map 900. In another instance, the previously determined first and second position vector is based on a parameter of the electrophysiological procedure, such as all or some correction vectors in a particular anatomical location, or all or some correction vectors at a particular time period, or a combination of parameters such as all or some correction vectors in a particular anatomical location (such as the location closest the current location of the catheter assembly 400) and all or some correction vectors at a particular time period, such as the most recent time period. Examples of combining or uniting correction vectors to obtain a previously determined first and second position vector can include averaging vectors or applying a weighted average to vectors, such as applying more weight to correction vectors most recent in time and less weight to correction vectors earlier in time.

In the example, the historical correction based on the previously determined first and second position vector at 704 is an estimate of the translation correction. The estimate is based on one or more prior correction vectors 800 as related to the first and second positions of the catheter elements. Applying the historical correction to the first and second positions generates device models having an initially corrected first and second positions at 704.

The device model of the catheter elements having initially corrected first and second positions are then subjected to the lateral alignment and longitudinal adjustments described above with respect to process 300. For example, the device model of the catheter elements having initially corrected first and second positions are corrected using translation, rotation, shape deformation, sheath detection, or other constraints, such as a constraint applied to the detected longitudinal movement to provide a longitudinal adjustment in process 300. In some instance, the generation of the initially corrected first and second position of the device models will create an accurate representation of the physical positions of the catheter elements, and no further constraints are applied during the sample. The application of the historical correction based on the previously determined first and second position vector to generate the initially corrected first and second positions of the device model provide for motion smoothing.

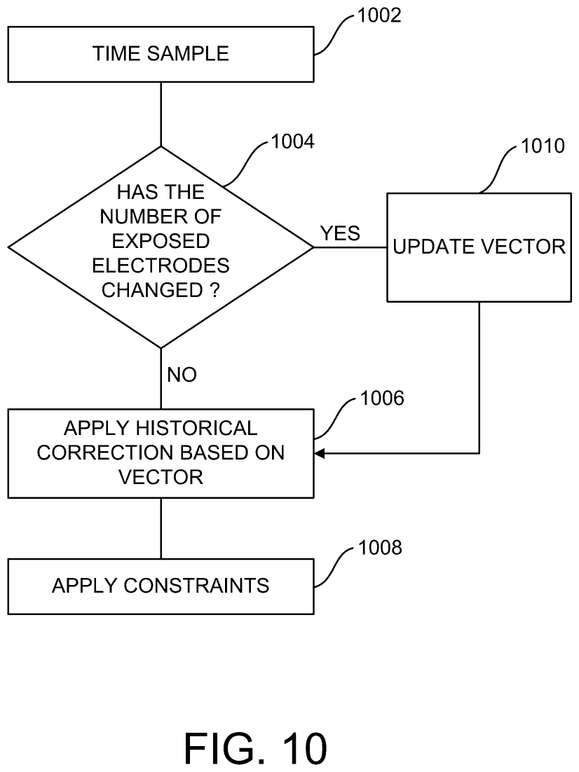

FIG. 10 illustrates an example flow chart 1000 of the process 700. At each time sample 1002, such as at each sampling period in process 700, a determination is made as to whether there is high confidence in a correction, such as the number of exposed electrodes 412 as determined from an independent parameter, such as sheath detection, has changed from the previous time sample at 1004. If the number of exposed electrodes 412 has not changed at 1004, a historical correction based on a previously determined first and second position vector is applied to the first and second positions of the first and second catheter elements at 1006. For example, a previously determined first and second position vector based on one or more correction vectors 800 of the plurality of correction vectors 910 is applied to generate a device model having an initially corrected first and second positions at 1006. Subsequently, longitudinal adjustments are applied to device model of the initially corrected first and second position based on the constraints described with respect to process 300 at 1008.

If the number of exposed electrodes 412 has changed at 1004, the previously determined first and second position vector is updated with a new correction vector 800 at 1010. Additionally, the correction map may be updated when the number of exposed electrodes changes by one, or whenever the computed corrections have high confidence. Additionally, the correction map may be understood to provide only partial corrections, depending upon the context, such as proximity of the assembly in the chamber relative to the spatial locations at which the correction map was able to be updated. Subsequently, a historical correction based on the updated previously determined first and second position vector is applied to the first and second positions of the first and second catheter elements at 1006. Translation corrections are applied to device model of the initially corrected first and second position based on the constraints described with respect to process 300 at 1008.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for use with an electrophysiological procedure, the system comprising:
a catheter assembly including a plurality of coaxially disposed catheter elements, the plurality of catheter elements including a first catheter element and a second catheter element, the first catheter element forming an elongated lumen defining a longitudinal axis and the second catheter element disposed within the lumen, the first and second catheter elements movable with respect to each other along the longitudinal axis, wherein the first catheter element includes a first tracking sensor configured to generate a first electrical signal and the second catheter element includes a second tracking sensor configured to generate a second electrical signal; and
a controller configured to:
generate a first position of the first catheter element based on the first electrical signal and generate a second position of the second catheter element based on the second electrical signal;
in response to a detected longitudinal movement of the second catheter element along the longitudinal axis with respect to the first catheter element:
apply a historical correction to the first and second positions based on a previously determined first and second position vector to obtain an initially corrected first and second positions;
apply a longitudinal adjustment to the initially corrected first and second positions based on a constraint applied to the detected longitudinal movement; and
generate an anatomical map of an organ of the electrophysiological procedure with a visualization of the catheter assembly including the longitudinal adjustment.

2. The system of claim 1, wherein the controller is configured to update the previously determined first and second position vector in response to the detected longitudinal movement of the second catheter element along the longitudinal axis with respect to the first catheter element.

3. The system of claim 2, wherein the updated previously determined first and second position vector is based on a plurality of previously determined first and second position vectors.

4. The system of claim 2, wherein the update to the previously determined first and second position vector is based on a parameter of the electrophysiological procedure.

5. The system of claim 2, wherein the update to the previously determined first and second position vector is based on a time period of the electrophysiological procedure.

6. The system of claim 2, wherein the update to the previously determined first and second position vector is based on an anatomical location of the electrophysiological procedure.

7. The system of claim 2, wherein the update to the previously determined first and second position vector is based on a plurality of parameters of the electrophysiological procedure.

8. The system of claim 1, wherein the controller is configured to laterally align the first position with the second position based on a lateral displacement and a rotational displacement determined from the first position and the second position.

9. The system of claim 8, wherein the first catheter position includes a first location in space and a first tangent, and the second catheter position includes a second location in space and a second tangent, and wherein the first location and the first tangent are laterally aligned with the second location and the second tangent based on a lateral departure and a rotational deflection determined from the first tangent and the second tangent.

10. The system of claim 1, wherein the controller is configured to track the first catheter element via impedance tracking and to track the second catheter element via magnetic tracking.

11. The system of claim 1, wherein the controller is configured to laterally align the second position to the first position.

12. The system of claim 1, wherein the controller is configured to apply the longitudinal adjustment to the second position with respect to the first position.

13. The system of claim 1, wherein the detected longitudinal movement of the second catheter element along the longitudinal axis with respect to the first catheter element is based on an independent parameter that detects the relationship between the sheath and electrodes on the catheter.

14. The system of claim 13, wherein the independent parameter includes sheath detection mechanism.

15. The system of claim 1, wherein the first catheter element includes a catheter sheath.

16. The system of claim 1, wherein the second catheter element includes an ablation catheter.

17. A system for use with an electrophysiological procedure, the system comprising:
a catheter assembly including a plurality of coaxially disposed catheter elements, the plurality of catheter elements including a first catheter element and a second catheter element, the first catheter element forming an elongated lumen defining a longitudinal axis and the second catheter element disposed within the lumen, the first and second catheter elements movable with respect to each other along the longitudinal axis, wherein the first catheter element includes a first tracking sensor configured to generate a first electrical signal and the second catheter element includes a second tracking sensor configured to generate a second electrical signal; and
a controller configured to:
generate a first position of the first catheter element based on the first electrical signal and generate a second position of the second catheter element based on the second electrical signal;
in response to a detected longitudinal movement of the second catheter element along the longitudinal axis with respect to the first catheter element based on a sheath detection:
apply a historical correction to the first and second positions based on a previously determined first and second position vector to obtain an initially corrected first and second positions;
apply a longitudinal adjustment to the initially corrected first and second positions based on a constraint applied to the detected longitudinal movement; and
generate an anatomical map of an organ of the electrophysiological procedure with a visualization of the catheter assembly including the longitudinal adjustment.

18. The system of claim 17, wherein the controller is configured to update the previously determined first and second position vector in response to the detected longitudinal movement of the second catheter element along the longitudinal axis with respect to the first catheter element and wherein the updated previously determined first and second position vector is based on a plurality of previously determined first and second position vectors.

19. A system for use with an electrophysiological procedure, the system comprising:
a catheter assembly including a plurality of coaxially disposed catheter elements, the plurality of catheter elements including a first catheter element and a second catheter element, the first catheter element forming an elongated lumen defining a longitudinal axis and the second catheter element disposed within the lumen, the first and second catheter elements movable with respect to each other along the longitudinal axis, wherein the first catheter element includes a first tracking sensor configured to generate a first electrical signal and the second catheter element includes a second tracking sensor configured to generate a second electrical signal; and
a controller configured to:
track the first catheter element via impedance tracking and to track the second catheter element via magnetic tracking;
generate a first position of the first catheter element based on the first electrical signal and generate a second position of the second catheter element based on the second electrical signal;
in response to a detected longitudinal movement of the second catheter element along the longitudinal axis with respect to the first catheter element based on a sheath detection:
apply a historical correction to the first and second positions based on a previously determined first and second position vector to obtain an initially corrected first and second positions;
apply a longitudinal adjustment to the initially corrected first and second positions based on a constraint applied to the detected longitudinal movement; and
generate an anatomical map of an organ of the electrophysiological procedure with a visualization of the catheter assembly including the longitudinal adjustment.

20. The system of claim 19, wherein the controller is configured to apply the longitudinal adjustment to the second position with respect to the first position.
